(19) United States
(12) Patent Application Publication
(10) Pub. No.: US 2022/0354670 A1
(43) Pub. Date: Nov. 10, 2022

(54) PROSTHETIC DEVICE FOR A LOWER EXTREMITY, ADJUSTING DEVICE FOR A PROSTHETIC DEVICE, AND METHOD FOR MANUAL ADJUSTMENT

(71) Applicant: OTTOBOCK SE & CO. KGAA, Duderstadt (DE)

(72) Inventors: Sven KALTENBORN, Duderstadt (DE); Miclas SCHWARTZ, Göttingen (DE); Dries GLORIEUX, Lommel (BE); Juan Pablo MEJA NINO, Mödling (AT); Gregory SCHNEIDER, Salt Lake City, UT (US)

(73) Assignee: OTTOBOCK SE & CO. KGAA, Duderstadt (DE)

(21) Appl. No.: 17/624,197

(22) PCT Filed: Jun. 30, 2020
(86) PCT No.: PCT/EP2020/068367

§ 371 Date: Dec. 30, 2021

(30) Foreign Application Priority Data
Jul. 4, 2019 (DE) 10 2019 118 118.6

(51) Int. Cl. A61F 2/76; A61F 2/66; A61F 2/70

(52) U.S. Cl. CPC A61F 2/76; A61F 2/6607; A61F 2/70; A61F 2002/705; A61F 2002/7635; A61F 2002/764; A61F 2002/7645

(57) ABSTRACT

The invention relates to a prosthetic device for a lower extremity comprising a prosthetic food and a lower leg part secured to the prosthetic foot, as well as a device for manually adjusting an orientation of the lower leg part relative to the prosthetic foot, wherein an inertial angle sensor is arranged on the prosthetic device, which detects the orientation of the lower leg part in the space and which is coupled to an output device which in turn outputs the orientation of the lower leg part in the space or the reaching of a previously determined orientation with an output signal in a manner that can be perceived by a user.

The invention relates to a prosthetic device for a lower extremity, comprising a prosthetic foot and a lower-leg part fastened to the prosthetic foot, and a device for manually adjusting an orientation of the lower-leg part relative to the prosthetic foot. The invention likewise relates to an adjustment device for manually adjusting an orientation of a lower-leg part relative to a prosthetic foot, of a prosthetic device, of a lower extremity, and a method for manually adjusting an orientation of a lower-leg part, of a prosthetic device, of a lower extremity relative to a prosthetic foot fastened to the lower-leg part, wherein an adjustment device with an inertial angle sensor is arranged on the prosthetic device, the inertial angle sensor detecting the orientation of the lower-leg part in space and being coupled to an output device.

Prostheses replace missing or lost limbs, the intention generally being that the prosthesis replaces not only the shape but also at least some of the functions of the limb. Prosthetic devices of the lower extremity comprise a foot part, the prosthetic foot, which is secured to the patient. Provided the lower leg or part of the lower leg is still intact, the prosthetic foot can be secured to the respective stump via a lower-leg socket. The lower-leg socket can be secured to the stump in various ways, for example using a prosthetic liner and suction socket technology. If the natural knee joint has been lost, the prosthetic device is generally secured to a thigh stump via a thigh socket. Then, a prosthetic knee joint is secured to the thigh socket, the prosthetic knee joint having a lower-leg tube or a lower-leg part for coupling the prosthetic foot to the prosthetic knee joint. Damping devices, adjusting devices and sensors and control devices, for example for controlling the damping device for the purposes of influencing the prosthetic knee joint, may be arranged on or in the lower-leg part.

The prosthetic foot can be mounted on the lower-leg part in articulated and motor-driven fashion in order to assist the patient with the respectively envisaged movement. Coordinating the movements in the knee joint and the ankle joint is very complicated in such an embodiment, and moreover the respective drive requires much space and is comparatively heavy. In the simplest embodiment of a prosthetic foot, the latter is embodied as a prosthetic foot without joints and—once it has been aligned—it is permanently secured to the lower-leg part. In this case, it is difficult to adapt the alignment of the prosthetic foot to different heel heights when the patient changes footwear. Furthermore, prosthetic feet that are mounted to be pivotable about an ankle joint and have a passive damping device to influence dorsiflexion or plantar flexion are known. Furthermore, there are prosthetic feet with a foot part and a proximal connection means, which is pivotably connected to the football. By way of an adjusting device, the foot part is adjustable relative to the connection means. The adjusting device can be assigned at least one position sensor which is coupled to a signal generation element, the latter outputting a signal regarding the attainment of the position of the foot part on the basis of the signal of the position sensor. The position sensor ascertains the relative position of the foot part with respect to the connection means or the lower-leg part fastened thereto. Alternatively, the position sensor ascertains the relative spatial position of the foot part during an adjustment procedure. Such a prosthetic foot is known from DE 10 2014 010 938 A1. As a result, it is possible to retrieve heel height settings.

A disadvantage of the device proposed in the prior art is that the correct position must be set individually for each shoe since it is only the relative angle between prosthetic foot and lower leg that is ascertained. For a new heel height it is therefore necessary to initially ascertain the correct setting and store the latter as a reference. Moreover, the user must identify the signal fitting to the respective heel height from a multiplicity of reference signals, possibly leading to incorrect settings.

It is therefore an object of the present invention to provide a prosthetic device, an adjustment device and a method for adjusting an orientation of a lower-leg part of a prosthetic device, which is simple to apply and which provides the user with reliable feedback about the correct prosthesis set up.

According to the invention, this object is achieved by a prosthetic device having the features of the main claim, and by an adjustment device and a method having the features of the alternative independent claims. Advantageous embodiments and developments of the invention are disclosed in the dependent claims, the description and the figures.

The prosthetic device for a lower extremity, comprising a prosthetic foot and a lower-leg part fastened to the prosthetic foot, and a device for manually adjusting an orientation of the lower-leg part relative to the prosthetic foot provides for an inertial angle sensor to be arranged on the prosthetic device, to serve to detect the orientation of the lower-leg part in space and to be coupled to an output device which outputs, in a manner identifiable by a user by way of an output signal, the orientation of the lower-leg part in space or the attainment of an orientation defined in advance. The output device preferably outputs, in a manner identifiable by a user, for example in optical, acoustic or tactile fashion, the defined orientation of the lower-leg part in space and thus provides feedback about the orientation of the lower-leg part in space. The output can be qualitative and/or quantitative, or else specify by way of a signal that an orientation of the lower-leg part in space, having been stored once, has been attained. Instead of measuring the orientation of the prosthetic foot in space and comparing whether this orientation corresponds to the desired orientation, it is the relative spatial position of the lower-leg part that is detected and not the relative position of the lower-leg part in relation to the prosthetic foot. Using such a prosthetic device it is possible, following the assembly of an adjustable prosthetic foot on the lower-leg part, to optimally adjust the prosthetic device by an orthopedic technician, that is to say ensure an optimal prosthesis setup. The prosthesis setup is the positioning and orientation of the respective prosthesis components in relation to one another. The prosthesis setup is set individually for each patient and contributes significantly to the functionality of the prosthetic device and its acceptance by the patient. Once the prosthesis setup has been correctly set, the patient can easily retrieve the optimal prosthesis setup using the prosthetic device according to the invention when a different shoe model with a deviating heel height and a deviating sole rigidity is used. By adjusting the prosthetic foot relative to the lower part until the stored standard angle or reference angle of the lower part in space is attained, the optimal setting set by the orthopedic technician is easily retrieved, and the prosthetic foot can be fixed to the lower-leg part in this position following a change of footwear or the like. The combination of determination of the lower-leg angle by means of an inertial sensor or inertial sensors and an output device allows the heel height to be adjusted for any prosthetic foot which comprises a device for manually adjusting an orientation of the lower-leg part. Adjusting an orientation of the lower-leg part in this case is implemented in fully manual fashion, from unlocking the joint via adjusting the angle to renewed locking. Accordingly, no complicated mechatronic ankle joint is required. Rather, a simple and reliable option for each adjustable ankle joint is offered to the patient in relation to retrieving the correct adjustment for different heel heights. The orientation in space is in particular the orientation of the lower-leg part relative to the direction of gravity. By way of example, if the lower leg is within the sagittal plane, the orientation of the lower-leg part is defined by the forward inclination or backward inclination in the opposite direction, proceeding from the perpendicular. If the user of the prosthetic device unlocks the prosthetic foot, in particular unlocks the latter manually, the prosthetic foot can be moved relative to the lower leg part. This is advantageously implemented when the prosthetic foot is put on and the lower leg part is pivoted within the sagittal plane, for example about a pivot axis in the region of the ankle joint. A signal perceivable by the user is output when the correct position of the lower-leg part is attained.

A development of the invention provides for the inertial angle sensor and the output device to be combined as a module and installed in the prosthetic device or detachably fastened thereto. As a result, it is possible to subsequently attach the adjustment device to a prosthetic device or to undertake retrofitting and equip prosthetic devices not originally designed to this end with the module. In principle, provision is made for the inertial angle sensor to be seated at any position on the lower-leg part, for example quite distally, in order to detect the relative spatial position without falsification where possible. Preferably, the inertial sensor is proximal of the prosthetic foot and proximal of the ankle joint such that valuable installation space in the prosthetic foot can be saved. The output device can be arranged at an easily accessible or more easily perceivable position, for example on a lower-leg socket, a thigh socket or at a separate location that is independent of the prosthetic device. In the case of a configuration as a separate element, the output device can be designed as tag on a set of keys, in a pocket, as an armband or as an app on a cellular telephone such that the user can receive the feedback as conveniently as possible. Then, the inertial angle sensor is preferably coupled to the output device in wireless fashion, by radio or a similar data transfer process. In the case of a configuration as a module, in particular as a retrofittable module, the inertial angle sensor and the output device are permanently interconnected, in particular by means of a cable. In the case of a separate arrangement, the output device may be coupled to the inertial angle sensor only when required. Then, the inertial angle sensor is coupled to a transmission device, which may also be designed as part of a control device for the remaining prosthetic device, the transmission device being used to supply the output device with the corresponding signal about the position of the lower-leg part in space. Both the inertial angle sensor and the output device are preferably coupled to a control device in which the sensor data are evaluated. By way of example, a computer and a memory device are available in the control device in order to evaluate the sensor data, perform a comparison with a reference angle in space, and output a signal.

The inertial angle sensor can preferably be arranged on the lower-leg part. As an alternative thereto, provision is made for the inertial angle sensor to be fastened to an element of the prosthetic device arranged proximally thereto, for example to an upper part of a prosthetic knee joint or to a thigh socket. From the information about the relative spatial position of the proximal element in conjunction with an angle sensor which measures the position of the lower-leg part with respect to the thigh socket, it is possible to calculate the relative spatial position of the lower-leg part. Alternatively or in addition, the ascertainment of the lower-leg angle can be implemented with a previously defined relative angle between thigh and lower leg such that no angle sensor is required. To this end, provision could be made for the user to always carry out or have to carry out the adjustment with the knee fully extended, for example.

A development of the invention provides for a load sensor to be arranged on the prosthetic device and be coupled to the output device in such a way that the output signal is output if a load is detected. The orientation of the lower-leg part may change in the case of different loads, in particular in the case of different axial loads. Different lower-leg orientations and hence a different prosthesis setup in each case may arise when a shoe is changed on account of different sole rigidities and sole geometries. By way of the load sensor it is possible to ensure that the adjustment is always implemented under the same load, e.g., axial load, as a result of which an unchanging prosthesis setup is ensured. The load sensor may be designed as an axial force sensor, pressure sensor or torque sensor and may be arranged, for example, in the lower-leg part, in a connecting device between the prosthetic foot and the lower-leg part, on a joint or on the prosthetic foot.

The output device is preferably designed to output an optical, acoustic and/or tactile output signal and can indicate the current relative spatial position of the lower-leg part, in particular in the sagittal plane, the deviation in a certain direction to the entered and stored orientation, and/or the attainment of the specified orientation. The output device may also be connected to, or integrated in, the device for manually adjusting an orientation of the lower-leg part. In this case, the user unlocks the adjusting device and moves the prosthetic foot manually until the reference position has been attained. A signal is output when the reference position is attained, as a result of which the user receives feedback that the correct setting was found.

Preferably, the prosthetic foot is mounted so as to be pivotable in the sagittal plane in order to compensate changes in the heel height. Provided a relative spatial position is also registered in the frontal plane, it is possible for example to output a warning signal should the deviation from a previously set value be too large.

A development of the invention provides for a deactivation device to deactivate the inertial angle sensor and/or the output unit or the connection between the inertial angle sensor and the output unit after the orientation defined in advance has been attained so that energy can be saved. The output unit preferably only operates when adjusting and fitting to a new shoe is intended. To this end, the prosthetic device can be put into an adjustment mode, for example by way of an input field in the output device or by way of another switch or command. Once adjustment and setting has been completed, this may either be detected automatically or be confirmed manually. The adjustment mode is then terminated, and the output device is deactivated. The inertial angle sensor can continue to be operated, for example in order to provide sensor data for a control device of any other prosthesis component, for example for controlling a prosthetic knee joint. Preferably, the inertial angle sensor is designed as part of a control device of the prosthetic device such that the relative spatial position data of the inertial angle sensor are not only used for identification and adjustment purposes when changing the heel height or changing footwear, but also serve when walking as a basis for, e.g., a change in a damping resistance in the ankle joint and/or the knee joint.

The adjustment device for manually adjusting an orientation of a lower-leg part relative to a prosthetic foot, of a prosthetic device, of a lower extremity provides for the adjustment device to comprise an inertial angle sensor which detects the orientation of the lower-leg part in space and which is coupled to an output device which outputs, in a manner identifiable by a user by way of an output signal, the orientation of the lower-leg part in space or the attainment of an orientation defined in advance. In particular, the output is implemented as an optical, acoustic and/or tactile output signal, wherein the output device in one variant of the invention is coupled to the inertial angle sensor and a computing device to form a module, for the purposes of evaluating the inertial angle sensor data and transmitting these to the output device.

A fastening device for securing to a prosthetic device can be arranged or formed on the adjustment device; by way of example, interlocking elements, such as clips, hook-and-loop fastener parts, screws, snap-fit elements and/or hooks, or frictionally connected elements such as magnets may be arranged or formed on the respective component in order to ensure a permanent or detachable and replaceable attachment. The attachment is preferably implemented on the lower-leg part or any other prosthesis component in a defined orientation, for example along an abutment edge or any other guide, for example on a rail or in a groove.

The method for manually adjusting an orientation of a lower-leg part of a prosthetic device to a lower extremity relative to a prosthetic foot fastened to the lower-leg part, wherein an adjustment device with an inertial angle sensor is arranged on the prosthetic device, the inertial angle sensor detecting the orientation of the lower-leg part in space and being coupled to an output device, provides for a reference orientation of the lower-leg part in space to be set for a user and the attainment of the reference orientation set in advance to be output in a manner identifiable by a user by way of an output signal. The reference orientation of the lower-leg part is preferably set by an orthopedic technician or another expert schooled to this end. The reference orientation is preferably input in the applied state of the prosthetic device, in the case of a reference setup for the prosthetic device in the case of a usual load. By way of example, a usual load is given by standing with a uniform weight load on the supported and unsupported side. In the case of such a method, it is possible to use the orientation of the lower-leg part in space as a relevant variable so that there is no need to store reference positions for different positions for each prosthetic foot. The user receives only a single signal, specifically once the reference orientation has been attained, optionally with notifications about the magnitude and the direction of the adjustment required in order to reach the reference orientation. The adjustment is implemented manually, in particular by pivoting about an ankle joint axis that extends perpendicular to the longitudinal extent of the lower-leg part in the frontal plane. This ensures that the lower-leg part is only pivoted in the sagittal plane. Alternatively, there can be pivoting on the fastening device of the foot part on the lower-leg part, for example at the so-called pyramid adapter, with pivoting in the frontal plane also being possible there as a matter of principle.

The orientation of the lower-leg part is preferably adjusted in the case of an applied, in particular loaded prosthetic device in order to be able to ensure for the user a prosthesis setup that remains unchanged during use, even in the case of different shoes.

The adjustment can be initiated and carried out automatically for each prosthetic foot change, for each heel height change or following a separate activation signal. By way of example, if a relative spatial position deviating from the reference orientation is detected following the application of the prosthetic device, a notification can be output to the user in respect of a reorientation or a check, and an adjustment or checking mode can be activated. Following the adjustment, the adjustment device preferably deactivates automatically; in particular, the output device is deactivated in order to minimize the power consumption. By way of example, if a shoe is changed, the output device provides an output signal or feedback that the lower-leg orientation currently present deviates from the reference orientation. Subsequently, the relative angle between the lower leg and prosthetic foot is adjusted manually until the output device provides the output signal or feedback that the lower-leg orientation corresponds to, or is sufficiently close to, the reference orientation. Once the correct position has been attained, the prosthetic foot is secured in this position, in particular by hand, such that the proximal connection means of the prosthetic foot, e.g., a pyramid adapter, no longer moves relative to the lower-leg part. The prosthetic foot itself may have a joint or move relative to the lower-leg part in regions. The end of the adjustment procedure may be indicated or output by way of the output device, for example after there was automatic recognition that the correct adjustment is present, or once a corresponding confirmation signal has been entered.

Exemplary embodiments of the invention are explained in more detail below on the basis of the attached figures, in which:

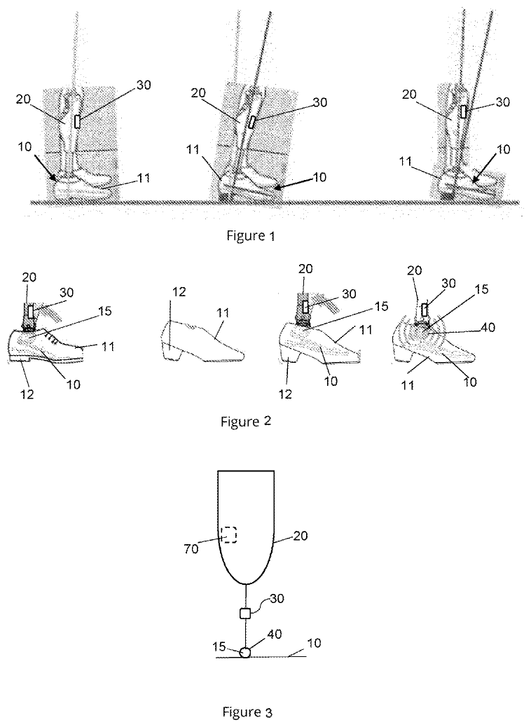

FIG. 1 shows a schematic illustration of an adjustment procedure;

FIG. 2 shows a prosthetic device with different shoes;

FIG. 3 shows a schematic illustration of a prosthetic device with a lower-leg socket;

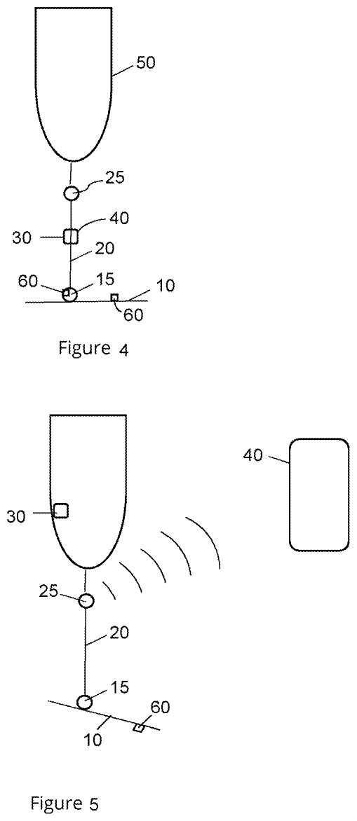

FIG. 4 shows a variant of FIG. 3 with a prosthetic knee joint and a thigh socket;

FIG. 5 shows a schematic illustration with a separate output device; and

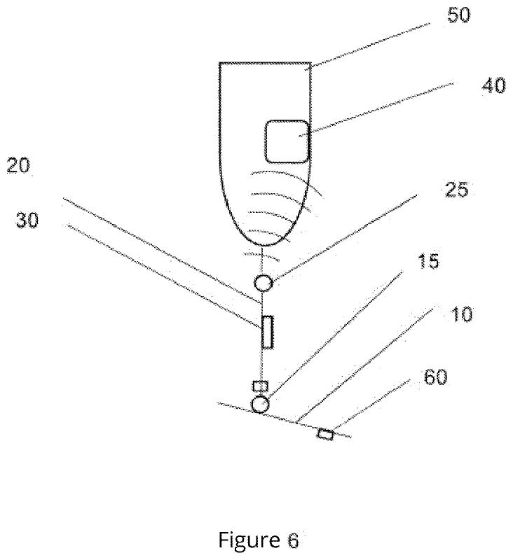

FIG. 6 shows a variant with an integrated output device.

FIG. 1 shows three positions or states in which a prosthetic device may be found during the use. In the left-hand illustration of FIG. 1, the prosthetic device is shown with a prosthetic foot 10 and a lower-leg part 20. A prosthetic knee joint which is connected to a thigh socket (not illustrated) is arranged at the proximal end of the lower-leg part 20. The prosthetic device is secured to a thigh stump by way of the thigh socket. In the illustrated exemplary embodiment, an inertial angle sensor 30, which is also referred to as inertial measurement unit or IMU and which may be constructed as an assembly made of one or more gyroscopes, optionally complemented by acceleration sensors, is arranged on the lower-leg part 20. The orientation shown in the left-hand illustration of FIG. 1 is stored as a reference orientation, with the respective longitudinal extents of the prosthetic foot 10 and of the lower-leg part 20 lending themselves as reference variables. The stored reference orientation is the so-called reference setup of the prosthetic device, which is set, stored and documented by an orthopedic technician. Storage may be implemented in a memory device which can be part of a control device for controlling a damping device in the prosthetic knee joint. Likewise, the inertial angle sensor 30 may be part of the control device for the prosthetic knee joint. The prosthesis setup is the spatial assignment of the individual prosthesis components to one another. What is sought after in the reference setting is that all prosthesis components are aligned optimally with respect to one another so that the prosthesis user can draw the greatest possible use from the prosthetic device. Since the prosthetic device is generally worn with a shoe 11, it is necessary to set the prosthesis setup when the shoe 11 is worn. As a rule, the shoe 11 is a model as usually worn by the user. If the shoe model is changed and if the shoe 11 has a different heel height, as illustrated in the middle illustration of FIG. 1, there is a change in the prosthesis setup and, in particular, in the orientation of the lower-leg part 20. In the middle illustration of FIG. 1, it is possible to identify that the longitudinal extent of the lower-leg part 20 is inclined forward on account of the different heel height, there likewise being a change in the inclination of the prosthetic foot 10. The inertial angle sensor 30 or the IMU 30 detect the inclination and the orientation of the lower-leg part 30 in space, either following the activation of a checking mode by the user or automatically. Since the orientation of the lower-leg part 30 no longer corresponds to the reference orientation, the signal that the alignment and the prosthesis setup are no longer correct is output via an output device (not illustrated). Subsequently, e.g., a locking of a pivot axis, about which the prosthetic foot 10 can be pivoted relative to the lower-leg part 20, is unlocked or released and the lower-leg part 20 is pivoted until the reference orientation has been resumed. As soon as this is the case, the output device provides an appropriate signal to the user, who can reactivate the locking device and lock the pivot axis. The correct relative spatial position is detected by way of the IMU or the inertial angle sensor 30 and indicated by an optical, acoustic and/or tactile signal. Alternatively, the respectively adopted relative spatial position angle or the distance from the reference angle or from the reference orientation can be indicated by way of the output device. The lower-leg part 20 is back in the original reference orientation in the right-hand illustration, as indicated by the dashed line.

FIG. 2 shows the relevant components of the prosthetic device in individual depictions. The left-hand illustration shows a prosthetic foot 10 in a shoe 11 with a heel 12. The prosthetic foot 10 has a pivoting device 15, about which the lower-leg part 20 in the form of a lower-leg tube can be pivoted about a pivot axis. An inertial angle sensor 30 is fastened to the lower-leg part 20. The second illustration from the left shows an alternative shoe 11 with a higher heel 12. In the third illustration from the left, the prosthetic foot 10 has been inserted into the alternative shoe 11. On account of the different heel drop between the two shoe models it is necessary to pivot the lower-leg part 20 counter to the opposite direction, that is to say to the back. To this end, the lower-leg part 20 is pivoted backward in the direction of the arrow, and the pivoting procedure or the relative spatial position orientation of the lower-leg part 20 is checked by the inertial angle sensor 30. As soon as the correct alignment of the lower-leg part 20 in space has been attained, in particular when the patient uniformly loads both the supported side and the unsupported side, an optical, acoustic and/or tactile signal is output by way of an output device 40, said signal indicating that the correct orientation has been attained. Then, the user locks the pivoting device 15 in relation to an unwanted displacement of the prosthetic foot 10 relative to the lower-leg part 20. Subsequently, the power supply to the output device 40 can be interrupted in order to save power. The inertial angle sensor 30 can continue to be used for the provision of sensor data.

In FIG. 3, the prosthetic device with the prosthetic foot 10, the lower-leg part 20 and the inertial angle sensor 30 fastened thereto is shown in a schematic illustration. The prosthetic foot 10 is mounted on the lower-leg part 20 so as to be pivotable about an axis by way of the pivoting device 15. The lower-leg part 20 has a lower-leg socket and a lower-leg tube, on which the inertial angle sensor 30 is secured either permanently or in removable fashion. Moreover, a securing device 70 is provided on the lower-leg socket, it being possible to secure the output device 40 (not shown) or a module consisting of the inertial angle sensor 30 and the output device 40 to said securing device. The module, the output device 40 and/or the inertial angle sensor 30 are designed to be securable and attachable to the securing device 70 in preferably nondestructively detachable fashion. Securing can be implemented by way of interlocking elements such as screws, bolts, hooks or clip elements, or by way of a frictional connection by means of magnets or by means of a combination of interlocking elements and frictionally connected elements. The arrangement of the inertial angle sensor 30 or the IMU then can be implemented either integrated in, or detachably fastened to, a part of the ankle joint, which is securely connected to the lower-leg part 20. It is likewise possible to arrange the sensor 30 on a structural part of the lower-leg part 20, for example the lower-leg tube or in the lower-leg socket.

FIG. 4 schematically illustrates a prosthetic device, in the case of which a proximal prosthesis component 50 is arranged on the lower-leg part 20. By way of example, the proximal prosthesis component 50 is a thigh socket with a connecting tube to a prosthetic knee joint 25. In the illustrated exemplary embodiment, the inertial angle sensor 30 is arranged again on the lower-leg part 20, alternatively the inertial angle sensor 30 and optionally the output device 40, too, can be arranged integrated in, or detachably fastened to, the knee joint 25 or the thigh socket or a connecting part between the thigh socket and the prosthetic knee joint. Likewise, the inertial angle sensor 30 can be arranged on an upper part of a prosthetic knee joint. In conjunction with an angle sensor which records the angle between the lower-leg part 20 and the proximal component 50, the relative spatial position of the lower-leg part 20 can be detected from the relative spatial position of the proximal component 50. In the exemplary embodiment of FIG. 4, load sensors 60 are moreover provided and arranged on the prosthetic foot 10 and the pivoting device 15. By way of example, the load sensors 60 can be axial force sensors, pressure sensors and/or torque sensors for registering the respective load on the prosthetic device. The load sensors 60 or the load sensor 60 are/is coupled to a control device which is also coupled to the inertial angle sensor 30. In this way, it is possible to recognize, for example, whether or not there is an adjustment of the prosthesis setup in the case of a loaded prosthetic device. In the exemplary embodiment as per FIG. 4, the output device 40 is combined in a module with the inertial angle sensor 30 and secured to the lower-leg part 20.

In FIG. 5, the output device 40 is formed separately and spatially separated from the inertial angle sensor 30, for example in the form of a cellular telephone to which appropriate data can be transmitted wirelessly from the inertial angle sensor 30, optionally by way of a separate transmission device. The data transfer from the sensor system to the output device 40 can be implemented by radio, WLAN, Bluetooth, NFC or any other method of transfer. The output device may output acoustic feedback or a vibration signal in addition to an optical display in order to inform the user about the correct adjustment following a heel height change.

FIG. 6 illustrates a further variant of the invention, in which the output device 40 is arranged on the prosthetic socket 50 ss a proximal component. The output device 40 is integrated in the prosthetic socket 50. The inertial angle sensor 30 or the IMU is arranged on the lower-leg part 20. The transfer from the inertial angle sensor 30 to the output device 40 is implemented wirelessly. Following manual unlocking of the prosthetic foot 10 relative to the lower-leg part 20, the prosthetic foot 10 is placed on the floor, for example with a shoe, in particular with a shoe with a heel height that differs from that of a previously fitted shoe. Contact between the prosthetic foot 10 and the floor is ascertained by way of the force sensor 60. The lower-leg part 20 is pivoted about the ankle joint 15 until the previously set reference orientation of the lower-leg part 20 in space has been attained. To this end, provision can be made, for example, for the lower-leg part 20 to be situated within the sagittal plane or else within a defined angular range medially and laterally from the sagittal plane. Once the reference orientation of the lower-leg part 20 has been attained, the output device 40 outputs an optical, acoustic or tactile signal, which is implemented when the desired position is attained.

1. A prosthetic device for a lower extremity, comprising a prosthetic foot and a lower-leg part fastened to the prosthetic foot, and a device for manually adjusting an orientation of the lower-leg part relative to the prosthetic foot, characterized in that an inertial angle sensor is arranged on the prosthetic device, serves to detect the orientation of the lower-leg part in space and is coupled to an output device which outputs, in a manner identifiable by a user by way of an output signal, the orientation of the lower-leg part in space or the attainment of an orientation defined in advance.

2. The prosthetic device of claim 1, wherein the inertial angle sensor and the output device are combined as a module and integrated in the prosthetic device or are detachably fastened thereto.

3. The prosthetic device of claim 1, wherein the inertial angle sensor is arranged on the lower-leg part or wherein the prosthetic device has a prosthesis component arranged proximal to the lower-leg part, and the inertial angle sensor is arranged on said prosthesis component.

4. The prosthetic device of claim 1, wherein a load sensor is arranged on the prosthetic device and is coupled to the output device in such a way that the output signal is output if a load is detected.

5. The prosthetic device of claim 4, wherein the load sensor is designed as an axial force sensor, pressure sensor or torque sensor.

6. The prosthetic device of claim 1, wherein the output device is designed to output an optical, acoustic and/or tactile output signal.

7. The prosthetic device of claim 1, wherein the prosthetic foot is mounted so as to be pivotable in the satittal plane.

8. The prosthetic device of claim 1, characterized by a deactivation device, which deactivates the inertial angle sensor and/or the output device or the connection between the inertial angle sensor and the output device after the orientation defined in advance has been attained.

9. An adjustment device for manually adjusting an orientation of a lower-leg part relative to a prosthetic foot of a prosthetic device of a lower extremity, wherein the adjustment device comprises an inertial angle sensor which detects the orientation of the lower-leg part in space and which is coupled to an output device which outputs, in a manner identifiable by a user by way of an output signal, the orientation of the lower-leg part in space or the attainment of an orientation defined in advance.

10. The adjustment device of claim 9, wherein the output device is designed to output an optical, acoustic and/or tactile output signal.

11. The adjustment device of claim 9, wherein a fastening device for securing to a prosthetic device is arranged or formed on the adjustment device.

12. A method for manually adjusting an orientation of a lower-leg part of a prosthetic device of a lower extremity relative to a prosthetic foot fastened to the lower-leg part, wherein an adjustment device with an inertial angle sensor is arranged on the prosthetic device, the inertial angle sensor detecting the orientation of the lower-leg part in space and being coupled to an output device, wherein a reference orientation of the lower-leg part in space is set for a user and the attainment of the reference orientation set in advance is output in a manner identifiable by a user by way of an output signal.

13. The method of claim 12, wherein the adjustment is performed in the case of an applied, or more particularly a loaded prosthetic device.

14. The method of claim 12, wherein the adjustment is carried out automatically for each change in prosthetic foot, for each change in heel height or following an activation signal.
